(19) United States
(12) Patent Application Publication
(10) Pub. No.: US 2012/0089012 A1
(43) Pub. Date: Apr. 12, 2012

(54) METHOD AND APPARATUS FOR NAVIGATING A CUTTING TOOL DURING ORTHOPEDIC SURGERY USING A LOCALIZATION SYSTEM

(75) Inventors: Wilhelm Baur, Schwarzenbruck (DE); Sabine Constanze Graf, Tuttlingen (DE); Jean-Baptiste Pinzuti, Gieres (FR)

(73) Assignee: Aesculap AG & CO. KG, Tuttlingen (DE)

(21) Appl. No.: 13/085,065

(22) Filed: Apr. 12, 2011

Related U.S. Application Data
(62) Division of application No. 11/254,027, filed on Oct. 18, 2005, now abandoned.

(51) Int. Cl. A61B 5/05

(52) U.S. Cl. 600/424

(57) ABSTRACT

The invention provides methods and apparatus for accurately cutting bones with a surgical cutting device, such as a sagittal saw, using a surgical navigation system without use of a complex cutting jig. A surgical navigation system is used to navigate a guide tube to be used to drill a k-wire into the bone. The k-wire will act as a guide to control at least one degree of freedom of a saw blade for making a cut in the bone. In an exemplary high tibial osteotomy procedure, in which two intersecting planar cuts must be made in the tibia in order to remove a wedge of bone, a surgical navigation marker is mounted on the guide tube. The surgeon uses the surgical navigation system to navigate the guide tube to the desired varus-valgus angle and height of the first cut and then drills a k-wire into the tibia at that varus-valgus angle using the guide tube. The process is repeated for the second cut. The surgeon then uses the two k-wires as guides for controlling the varus-valgus angle of a sagittal saw for the two planar cuts. The surgeon rests the saw blade flat on the respective k-wire to define the varus-valgus angle of the cut. The saw itself also is navigated, with the surgical navigation system providing a display showing the surgeon at least (1) the varus-valgus angle, (2) the cut depth, and (3) the anterior-posterior slope. The anterior-posterior slope and the depth of the cut is controlled freehand by the surgeon.

### FIELD OF THE INVENTION

The present invention relates to surgical navigation systems, often called localization devices. More particularly, the present invention relates to methods and apparatus for navigating a cutting tool during orthopedic surgery using a surgical navigation system.

### BACKGROUND OF THE INVENTION

In an exemplary surgical navigation system 100 such as illustrated in FIG. 1, at least two sensors 114a, 114b (e.g., infrared cameras) mounted in a housing 128 are used to detect a plurality of markers 116a, 116b, 116c, 116d, 116e that can be mounted on the patient's bones 105a, 105b and/or on surgical tools 124. More particularly, the cameras 114a, 114b are coupled to a computer 112 that analyzes the images obtained by the cameras and detects the positions and orientations of the various bones and/or tools bearing the markers during the surgery and calculates and displays useful information for performing the surgery to the surgeon on a monitor 122. The computer system may be provided in a portable cart 108 and may include a memory 110 for storing data and operational software, a keyboard 120, and/or foot pedals 118 for entering data.

One such surgical navigation system is the OrthoPilot available from Aesculap, Inc. of Center Valley, Pa., USA.

With reference to FIG. 2A, which is an enlarged view of an exemplary marker 116 mounted on a sagittal saw 202, each marker 116 comprises a base with a mounting mechanism 217 on one end for mounting to a complementary mounting mechanism 201 on a piece of medical equipment, such as sagittal saw 202 of FIG. 2A, surgical pointer 124 of FIG. 1, a bone screw, or a cutting jig. Extending from the other end of the base are at least three infrared LED transmitters 208. Alternately, instead of transmitters, the system could utilize markers 116a bearing infrared reflectors 208a, as shown in FIG. 2B, which marker 116 mounted on a sagittal saw 202, each marker 116 comprises a base with a mounting mechanism 217 on one end for mounting to a complementary mounting mechanism 201 on a piece of medical equipment, such as sagittal saw 202 of FIG. 2A, surgical pointer 124 of FIG. 1, a bone screw, or a cutting jig. Extending from the other end of the base are at least three infrared LED transmitters 208. Alternately, instead of transmitters, the system could utilize markers 116a bearing infrared reflectors 208a, as shown in FIG. 2B, which illustrates an exemplary marker 116a of the reflector type. When using reflectors, the surgical navigation system includes an infrared light source 107 (FIG. 1) directed towards the surgical field so that the reflectors 208a will reflect infrared light back to the two cameras 114a, 114b. With at least two cameras and at least three transmitters 208 (or reflectors 208a) per marker, sufficient information is available to the computer to determine the exact position and orientation of each marker 116 (or 116a) in all six degrees of freedom, i.e., the three translational degrees of freedom, x, y, and z and the three orientational degrees of freedom, i.e., rotation around each of the x, y, and z axes). With respect to a bone or other anatomical feature, the three translational degrees of freedom might be expressed in terms of height (along the mechanical axis of the bone), anterior-posterior position, and medial-lateral position and the three orientational degrees of freedom might be expressed in terms of varus-valgus angle, anterior posterior slope, and rotation about the mechanical axis of the bone.

The mounting mechanism at the end of the base of the marker is designed to mate with a complementary mounting mechanism on the surgical instrument in only one position and orientation. The computer is preprogrammed with information relating to the position and/or orientation of the operational portion(s) of the medical instrument relative to the marker when mounted on it. In this manner, by detecting the position and orientation of the marker, the computer will also know the position and orientation of the medical instrument and its operational portion(s). For instance, the medical instrument may be the pointer 124 shown in FIG. 1 having a tip 124a, the exact position of which is known relative to the marker 116a.

In most surgeries involving surgical navigation, it is necessary to discern two or more markers 116 or 116a from each other since two or more markers will be tracked simultaneously by the system. This can be done in several different ways. If LED transmitters are used, each transmitter 208 can be timed to emit light only during a specific time interval thpt the computer is preprogrammed to know is the time interval assigned to that particular transmitter on that particular marker. The LEDs are illuminated in sequence at a very high rate so that the computer has virtually continuous information as to the exact location of every LED. Alternately, when using reflectors, each marker 116a may have its three or more reflectors 208a positioned in slightly different positions relative to each other so that the computer can discern which marker it is observing by determining the geometric relationship between the three or more reflectors 208a on the marker 116a.

Referring back to FIG. 1, the markers 116 are fixedly mounted on bones 105 (via bone screws or pins) and or medical instruments 124 (FIG. 1) or 202 (FIG. 2A) positioned within the field of view of the cameras 114a, 114b so that the computer 112 can track the location and orientation of those bones and/or medical instruments. The computer will then generate useful information to help the surgeon determine appropriate locations or alignments for prosthetic implants, cutting jigs, saws for cutting bones, and the like and display it in a display 123 on the monitor 122.

One known use for surgical navigation systems is in leg surgery. In High Tibial Osteotomy, for instance, a surgeon must remove a wedge of bone from the patient's tibia near the knee joint in order to correct the patient's stance. In order to correct bow-leggedness (sometimes called 0-leggedness), a wedge of bone is removed in which the thick portion of the wedge is on the lateral side of the patient's leg and the thin or pointed portion of the wedge points medially. On the other hand, to correct a knock-kneed stance (sometimes called X-legged), the thick side of the wedge is medial and the pointed side is lateral.

FIGS. 3A and 3B are frontal views of a tibia illustrating the procedure for HTO for correcting bow-leggedness. Prior to surgery, the surgeon determines the exact angle of correction desired for the particular patient as well as the exact angle of the wedge to be removed. These angles are often the same angle. However, for reasons well understood by those working in the field of knee surgery, and particularly HTO surgery, these angles might be off by a degree or two in some cases. In FIG. 3A, lines 312 and 314 represent the varus-valgus angles of the two cuts that are to be made in the patient's tibia 316. Generally, one wants the anteriorposterior slope of the two cuts (which cannot be seen in the frontal view of FIGS. 3A and 3B) to be identical.

In cutting and removing the wedge, the surgeon does not cut completely through the tibia, but instead leaves a small portion of bone 318 adjacent the point of the wedge so that the tibia is still one piece. The small remaining portion of bone essentially is a hinge 318. The wedge of bone 320 is removed leaving a wedge shaped gap 320a as shown in FIG. 3B and hinge 318. Referring now to FIG. 3C, the distal portion of the tibia 316b is bent around the hinge 318 so as to close the gap created by the removed wedge 320. A bone plate 322 is then installed with bone screws 324 to hold the bone in the closed position, thus correcting the patient's stance.

The wedge cuts are made using a sagittal saw such as the one shown in FIG. 2A.

There are several known techniques for HTO surgery. In the Coventry technique, for instance, the first cut 312 is made parallel to the tibial plateau (i.e., perpendicular to the mechanical axis of the tibia) and is typically made about 10 mm below the tibial plateau. The second cut 314 is made at the desired wedge angle to the first cut 312 and, as noted above, meets the first cut approximately at an apex that should leave about 5 to 10 mm of bone (measured in the lateral-medial direction) to form the aforementioned hinge 318 and to prevent the tibia from being cut into two separate pieces. A typical angle for a lateral HTO might be in the 5-10° range. FIGS. 3A and 3B show an exemplary angle of 7°.

Typically, a surgeon precisely aligns the cuts using a cutting jig and a sagittal saw. First, the surgeon mounts a cutting jig having a thin slot just wide enough to accept the blade of the sagittal saw. The jig is precisely placed to define the varusvalgus angle, anterior-posterior slope, and height of the cut and is then fixedly mounted to the tibia with a plurality of bone screws or pins. The surgeon then cuts the bone with the sagittal saw by inserting the saw blade into the slot of the jig. The surgeon typically controls the depth of the cut manually, as the cutting jig does not control cut depth. Specifically, the surgeon might measure the lateral-medial width of the bone and then control the saw to make a cut about 5-10 mm less than that width. For example, the surgeon might draw a mark on the saw blade that will be visible in an X ray and then take an x-ray after some sawing when he or she thinks the saw blade is approaching the desired final depth. The surgeon may then saw more and repeat the process until he or she confirms that the saw has reached the desired cut depth.

Surgical navigation systems have been employed to help surgeons position the jig and to guide the saw. In one such technique, prior to the surgery, the surgeon inputs data to the surgical navigation system indicating the desired correction angle. The surgeon mounts markers on the tibia and the femur and the navigation system can be used to track those markers in order to track the position and orientation of the bones. The surgeon would then typically palpate various landmarks on the tibia and femur with a surgical pointer bearing another marker while the surgical navigation system records those points relative to the tibial or femoral marker. The surgical navigation system will then always know the location of those landmarks as a function of the position and orientation of the markers mounted to the tibia and femur, respectively. Such points typically include the patellar insertion point, the lateral and medial epicondyles, the lateral and medial plateaus, the lateral malleolus, the center frontal plane, and the tibial medial cortex.

From those points, the surgical navigation system can calculate other landmarks on the tibia such as the height of the tibial plateau, the lateral-medial width of the tibia approximately where the two wedge cuts are to be made.

The surgeon also may obtain kinematic data to define the mechanical axis of the tibia. One such technique for obtaining kinematic data and calculating the mechanical axis of the bone therefrom can be found in U.S. Pat. No. 6,385,475, incorporated herein by reference.

The navigation system can be used to track the marker mounted on the tibia in order to track the position and orientation of the bone (and its mechanical axis and other landmarks) as the leg is moved. Then, another marker can be mounted to a cutting jig for guiding the saw for cutting the wedge cuts. The navigation system can be used to display the position of the cutting jig relative to the various landmarks of the bone (which is being tracked via the marker mounted on the tibia) so that the surgeon can determine when the jig is positioned in exactly the desired orientation and position relative to the bone for making the cut. The surgeon can then affix the jig to the bone in that position and make the cut.

The surgeon must accurately position the cutting jig in at least three degrees of freedom. Particularly, the height, anterior-posterior slope, and varus-valgus angle of the cutting plane must be set very precisely relative to the mechanical axis of the bone. For instance, in the Coventry technique, the height of the cuts must be selected so that the lower cut is entirely above the patellar insertion point. Generally, the anterior-posterior slope of both cuts should be 0° relative to the tibial plateau (i.e., perpendicular to the mechanical axis of the tibia). However, more importantly, the anterior-posterior slope of both cuts should be precisely the same as each other to assure that the surfaces mate with each other when the wedge is closed. Finally, the varus-valgus angles of the two cuts relative to each other should be the desired wedge angle. In one exemplary surgical navigation system, to make the first wedge cut 312, the system shows on the computer monitor the orientation of the cutting plane of the cutting jig relative to the mechanical axis of the tibia in two planar views, namely, a frontal view (in which the varus-valgus angle of the cutting plane is visible), and an sagittal view (in which the anterior-posterior slope of the cutting plane is visible). It also shows the height of the cut in one of the views (preferably displayed as the distance from the patellar insertion point of the lowest point of the lower cut, which is a value calculated from the height and angle of the first cut and the known desired angle of the second cut relative to the first cut). The surgeon must manipulate the jig in these three degrees of freedom while looking at the monitor. The surgeon must then attach the cutting jig to the tibia with multiple bone screws or pins while holding the jig steady in this position.

The surgeon can then mount a surgical navigation system marker to a sagittal saw so the surgical navigation system can track the depth of the cut and display to the surgeon the distance of the front tip of the saw blade from the medial cortex. As previously noted, the surgeon typically will want to stop the cut about 5-10 mm short of the medial cortex. It may also track and display the varus-valgus and anteriorposterior slope of the saw blade, but these parameters are already precisely controlled by the cutting jig itself and therefore need not be tracked by the surgical navigation system.

Some surgeons find it difficult to position a jig accurately using surgical navigation systems because they must precisely position the cutting jig on the bone in multiple degrees of freedom while trying to look at both the computer monitor and the patient's knee, and then attach the jig to the bone with multiple pins using a power tool while not moving the jig.

It is an object of the present invention to provide an improved method and apparatus for surgical navigation.

It is another object of the present invention to provide an improved method and apparatus for cutting bones using a surgical navigation system.

It is a further object of the present invention to provide an improved method and apparatus for cutting a wedge for a high tibial osteotomy surgical procedure.

### SUMMARY OF THE INVENTION

The present invention provides methods and apparatus that overcome the aforementioned problems by permitting one to accurately cut bone with a surgical cutting device, such as a sagittal saw, using a surgical navigation system without use of a complex cutting jig. In accordance with a first aspect of the invention, a surgical navigation system is used to navigate a guide tube that will be used to drill a k-wire into the bone. The k-wire will act as a guide to control at least one degree of freedom of a saw blade for making a cut in a bone. In an exemplary high tibial osteotomy procedure, for example, in which two intersecting planar cuts must be made in the tibia in order to remove a wedge of bone, a surgical navigation marker is mounted on the guide tube. The surgeon uses the surgical navigation system to navigate the guide tube to the desired varus-valgus angle of the first cut and then drills a k-wire into the tibia at that varus-valgus angle using the guide tube. The drill itself also may be navigated for redundancy and extra accuracy. The same process is repeated with respect to the second cut. The surgeon can then use the two kwires as guides for controlling the varus-valgus angle of a sagittal saw for the two planar cuts. Particularly, the surgeon rests the saw blade flat on the respective kwire to define the varus-valgus angle of the cut. The saw itself also is navigated, with the surgical navigation system providing a display showing the surgeon at least (1) the varus-valgus angle, (2) the cut depth, and (3) the anterior-poster slope. The anterior-posterior slope and the depth of the cut is controlled freehand by the surgeon

### BRIEF DESCRIPTION OF THE DRAWINGS

FIG. 1 is an illustration of a surgical navigation system being used for knee surgery in accordance with the prior art.

FIG. 2A is a close-up perspective view of a marker of the LED emitter type for use with a surgical navigation system mounted on a surgical sagittal saw in accordance with the prior art.

FIG. 2B is a perspective view of a marker of the reflector type of the prior art.

FIGS. 3A-3C are drawings of a tibia illustrating the procedure involved in a lateral high tibial osteotomy that can be performed in accordance with the present invention.

FIG. 4 is an illustration of an exemplary k wire guide tube that may be used in connection with the present invention.

FIG. 5 is an illustration of a display screen for navigating a first k wire in accordance with one aspect of the present invention.

FIG. 6 is an illustration of a display screen for navigating a second k wire relative to the k wire in accordance with another aspect of the present invention.

FIG. 7 is a drawing illustrating a tibia after the k-wires have been installed in accordance with the present invention.

FIG. 8 is an illustration of a display screen for navigating the first bone cut in accordance with one aspect of the present invention.

FIG. 9 is an illustration of a display screen for navigating the second bone cut relative to the first bone cut in accordance with the present invention.

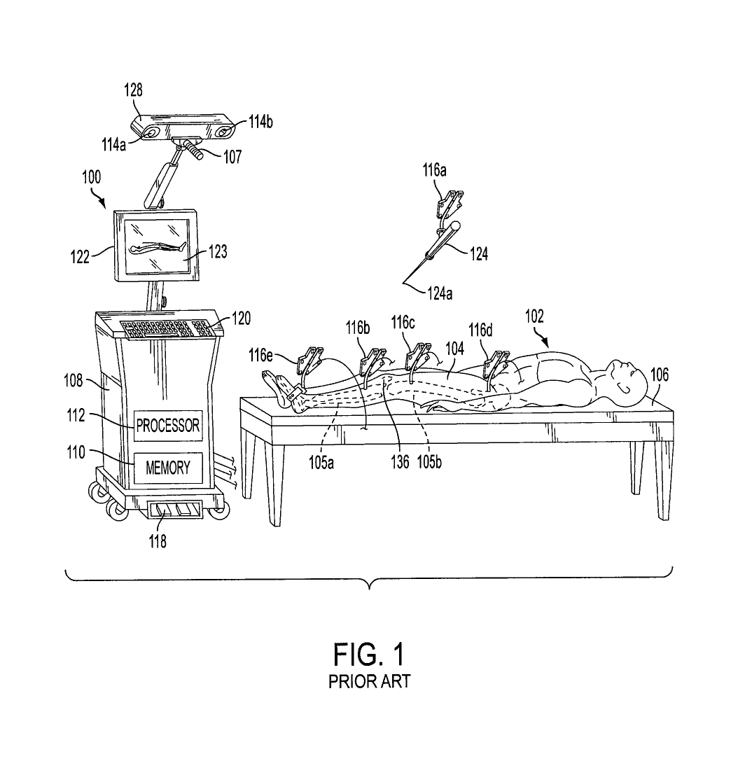

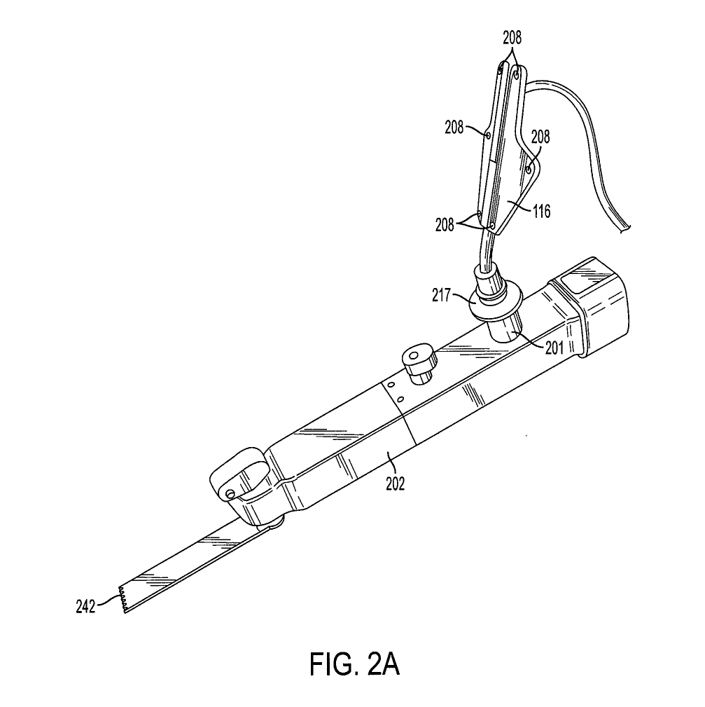

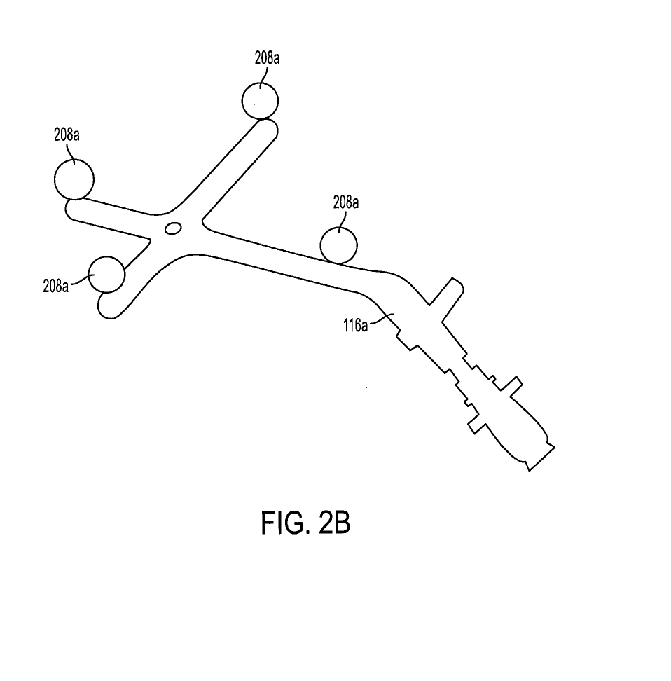

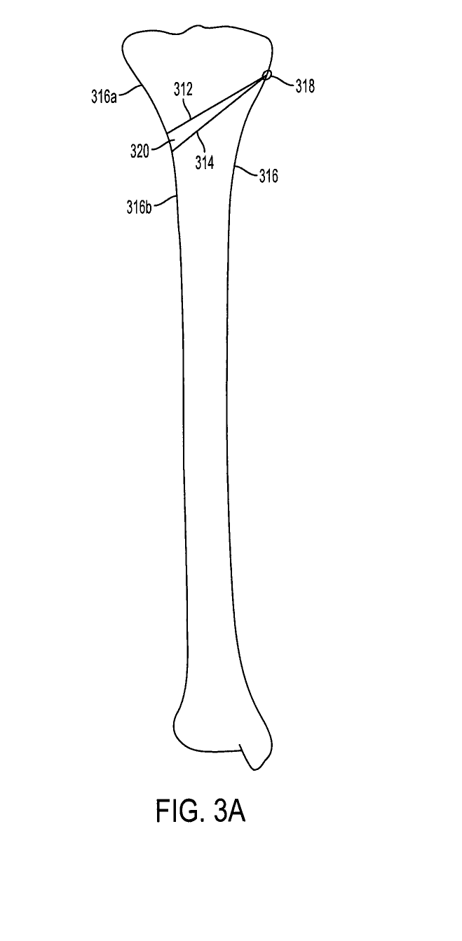

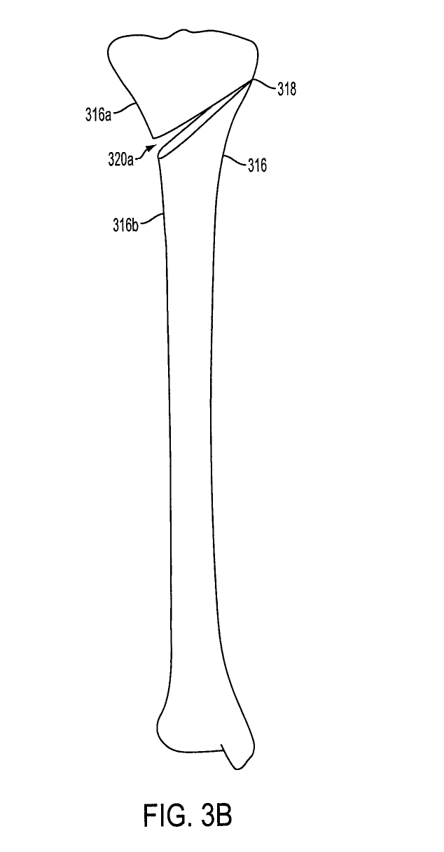

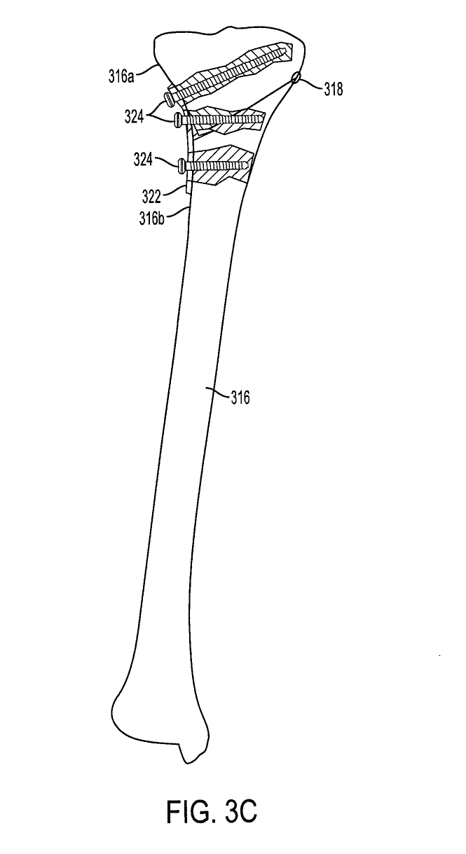

### DETAILED DESCRIPTION OF THE INVENTION

A description of a suitable localization device for use in connection with the present invention is found in U.S. Pat. No. 6,385,475 to Cinquin et al., incorporated herein by reference.

The present invention will be described in connection with an exemplary high tibial osteotomy (HTO) surgical procedure. However, it should be understood that the invention has broader applications and can essentially be applied to any bone cutting. As will become apparent from the discussion below, the technique in accordance with the present invention is simpler and less time consuming than the prior art techniques discussed above.

Prior to the surgery, the surgeon has determined both the desired correction angle and, if different, the desired angle of the wedge to be removed and this information is input into the memory of the surgical navigation system for use during the surgery, as will be described below.

The surgeon surgically opens the knee with standard incisions for HTO and mounts a marker on a surgical pointer that can be tracked by the surgical navigation system. The surgeon also mounts markers fixedly to the tibia and the femur, such as by affixing a bone screw or pin to the bone near the knee joint, and then mounting a marker on the bone screw. Then, the surgeon palpates various significant landmarks on the tibia and femur while the surgical navigation system records this information.

The surgical navigation system, of course, is programmed with information defining the location of the tip of the pointer relative to the position of the marker mounted on the pointer. The surgeon palpates the various relevant landmarks and then presses a pedal or otherwise indicates to the surgical navigation system to record that point as the particular landmark. In one embodiment of the invention, the display monitor displays information indicating to the surgeon the particular landmark that should be palpated. The surgeon can then touch the tip of the pointer to that particular landmark and press the input pedal in order to record it in the surgical navigation system. The screen will then change to indicate the next landmark to be palpated. The process continues until all of the relevant landmarks are palpated. For high tibial osteotomy, relevant points that might be palpated and recorded include the medial and lateral epicondyles, the medial and lateral tibial plateaus, the patellar insertion point, the medial and lateral malleoli, the center frontal plane, and the tibial medial cortex.

Typically, the medial cortex is palpated by drilling horizontally through the tibia from the lateral side about 10 mm below the tibial plateau and then palpating the medial cortex with a hooked pointer inserted through the drilled hole from the lateral side.

In addition, it is also desirable to determine the mechanical axis of the tibia kinematically, as mentioned above. This can be performed with a surgical navigation system by tracking the motion of the tibia as the knee joint is flexed. Techniques for determining the centers of the femoral head, ankle joint, and knee joint, and determining the mechanical axes of the tibia and/or femur therefrom are known and will not be described herein.

At this point, instead of navigating and mounting a cutting jig to the tibia for guiding the first cut, the surgeon navigates and mounts a k-wire in the tibia for defining only the desired varus-valgus angle for the first wedge cut.

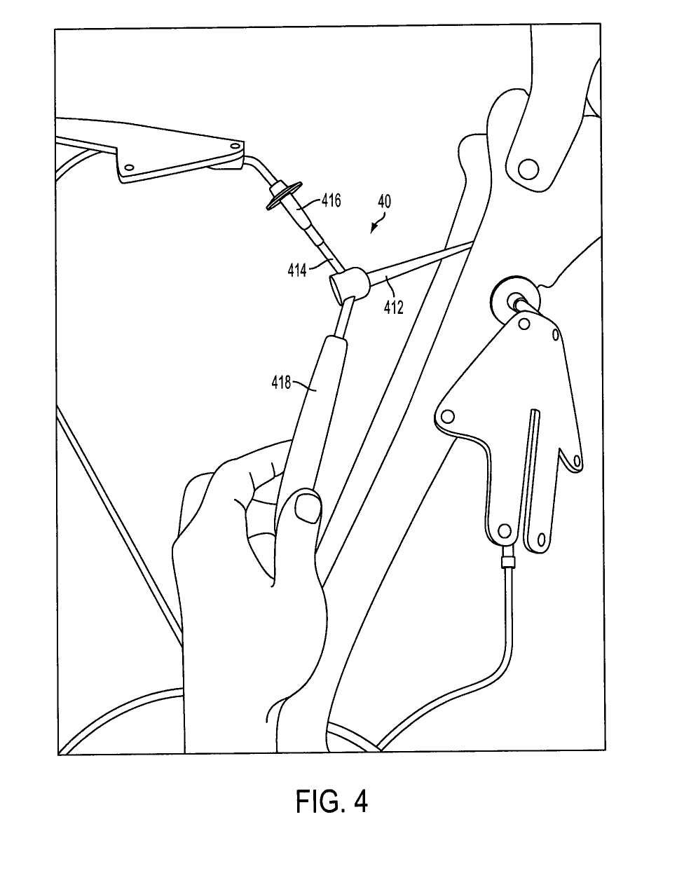

In one preferred embodiment of the invention, a marker trackable by the surgical navigation system is mounted on a guide tube that will be used to guide the drilling of the k wire. An exemplary guide tube 410 is shown in FIG. 4 and may be as simple as a hollow cylindrical tube 412 having an inner diameter slightly larger than the k-wire and a mounting mechanism 414 for mounting a marker 416 on the tube. The surgical navigation system, of course, is programmed with information defining the axis of the guide tube 410 relative to the position and orientation of the marker. A handle 418 may be disposed near the proximal end of the guide tube to facilitate handling of it by the surgeon.

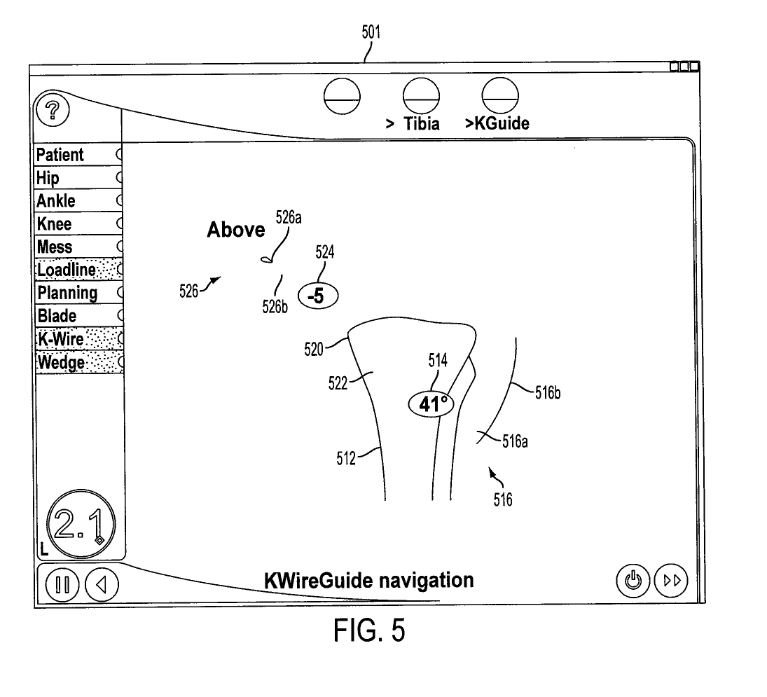

FIG. 5 is an illustration of an exemplary display screen 501 that a surgical navigation system in accordance with the present invention might provide in connection with navigating the first cut for the wedge in an HTO operation. The guide tube 410 is brought within the field of view of the surgical navigation system. The display screen 501 shows a pictorial representation of the tibia at 512. The representation may be a stock representation having no relationship to any of the actual measurements made of the tibia or, alternately, may be based on those measurements. The surgical navigation system detects the marker on the guide tube 410 as well as the marker on the tibia and can thus calculate and display in screen 501 relevant information as to the position and orientation of the guide tube 410 relative to the tibia. For instance, FIG. 5 shows several relevant pieces of information with respect to the first cut in the HTO bone wedge removal. The first piece of information shown is the varus-valgus angle of the guide tube (which will define the varus-valgus angle of the k-wire that is drilled into the bone, which, in turn, will define the varus-valgus angle of the cut of the saw as described below). The varus-valgus angle of the guide tube is measured relative to a plane parallel to the tibial plateau (i.e., a plane orthogonal to the mechanical axis of the tibia). This information is shown in screen 501 by the numerical angle appearing in oval 514. In addition, the same information is redundantly shown graphically by graphic 516 comprising line 516a and arc 516b. As the angle changes, the number in oval 514 changes accordingly. Likewise, the orientation of line 516a and the shading of arc 516b changes accordingly to represent changes in the varus-valgus angle of the guide tube 410.

The surgeon decides the desired angle for the first cut. Generally, there are two well-known techniques, namely, the Wagner technique, in which the first cut is made at approximately a 40° to 45° angle to the tibial plateau, and the Coventry technique, in which the first cut is made approximately parallel to the tibial plateau (i.e., 0°). FIG. 5 demonstrates a procedure in which the surgeon is using the Wagner technique.

Another piece of information displayed in screen 501 is the orthogonal distance between the longitudinal axis of the guide tube 410 and the point that was palpated on the medial cortex. This point is represented by red dot 520 and the longitudinal axis of the guide tube is represented by dashed line 522. The aforementioned orthogonal distance is displayed numerically and graphically. Specifically, it is displayed numerically in oval 524 and graphically at 526 by line 526a and arrow 526b. The two cuts should intersect about 5 to 10 mm laterally of the point palpated on the medial cortex so that they will meet there and remove a wedge while leaving a 5-10 mm millimeter wide hinge of bone.

In the numerical display 524, if the axis of the guide tube crosses above the point palpated on the medial cortex, the distance (in mm) is displayed as a negative number. If it crosses below the point palpated on the medial cortex, it is displayed as a positive number. The graphical display 526 also visually shows whether the axis is above or below the point palpated on the medial cortex. Even further redundantly, the screen may include the word “Above” or “Below” to indicate whether the axis of the guide tube crosses above or below the point palpated on the medial cortex.

Optionally, the display may also offer information as to the axial rotation of the guide tube relative to the mechanical axis of the tibia. The guide should be oriented in the medial-lateral plane. In a preferred embodiment, the display shows nothing as long as the guide is oriented within a predefined range of being in the medial-lateral plane (e.g., 10 degrees). However, if it is not within this range, the display presents a text message indicating such and preferably indicating the angular difference of the guide's longitudinal axis to the medial-lateral plane.

The surgeon guides the guide tube by hand to the desired angle and position relative to the point palpated on the medial cortex and then drills the k-wire into the bone using the guide tube as a guide. If desired, a marker can be mounted on the drill also and the drill can be navigated on the same or a different display screen. However, this is not necessary as it would merely provide redundant information to that provided by the navigation of the guide tube.

In an alternative embodiment of the invention, the guide tube may be dispensed with and the drill may instead be navigated directly.

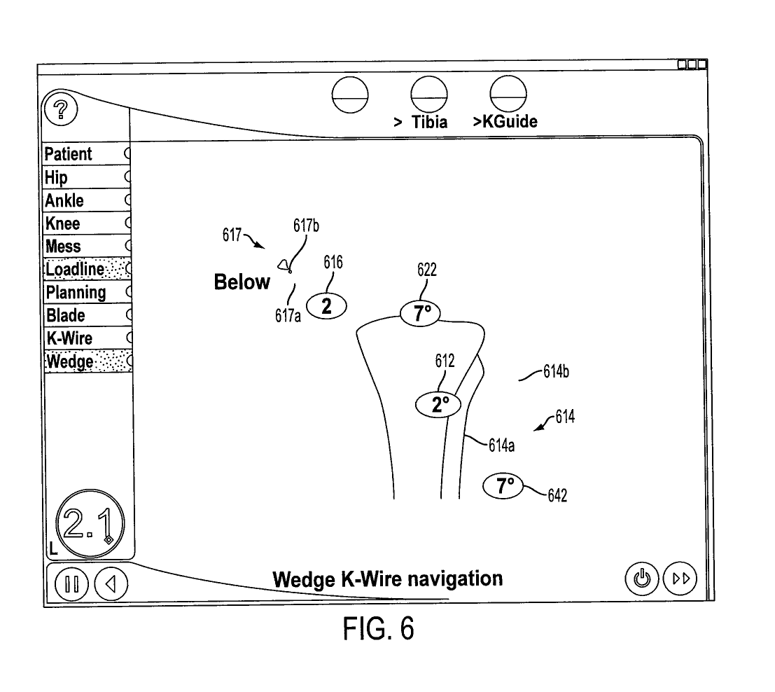

The k-wire should not be drilled completely through the bone. At this point, the surgeon will indicate to the surgical navigation system that he has completed insertion of the first k-wire and is now moving on to navigation of the second k-wire. This may be done, for instance, by tapping on a pedal, in response to which the software will simply move on to the screen shown in FIG. 6 which is used for navigating the second k-wire. The surgical navigation system will record in memory the position and orientation of the guide tube (and thus the k wire) at the instant the pedal is depressed.

The guide tube 410 can now be slipped off of the first, installed k-wire and used to navigate the second k wire using screen 600 in FIG. 6. This screen is very similar to the screen in FIG. 5 except that it includes additional information and presents some of the information in a different way. For instance, in FIG. 6, oval 612 numerically shows the angle of the guide tube 410 as measured from the angle of the first k-wire rather than from the tibial plateau. The same information is shown redundantly just like in screen 501 of FIG. 5 by graphical display portion 614, comprising line 614a and arc 614b. Oval 616 and associated redundant graphical display 617, comprising line 617a and line 617b essentially show exactly the same information as described above with respect to oval 524 and graphical display portion 526 in FIG. 5, namely the orthogonal distance between the longitudinal axis of the guide tube and the point palpated on the medial cortex.

Typically, the surgeon will want the orthogonal distance to the point palpated on the medial cortex to be the same for the first and second cuts so that the first and second cuts will intersect at that orthogonal distance from the point palpated on the medial cortex.

FIG. 6 additionally includes a shaded oval 622 that displays the desired correction angle that the surgeon had input into the system prior to the surgery. This information has previously been input and does not change during navigation. It is merely a reminder to the surgeon of the desired wedge angle.

Also displayed in shaded oval 642 is the computed wedge angle which, as previously noted, may or may not be the same as the desired correction angle.

In a preferred embodiment of the invention, ovals 622 and 642 are a different shade than the ovals that show real time data, such as ovals 612 and 616, in order to provide easy visual differentiation between the fixed display data that is merely a reminder and the real time tracked data. Also in a preferred embodiment, when the guide tube is positioned at an angle within a predetermined range of the desired angle (e.g., within 1°), oval 612 changes colors (e.g., white to green) to indicate proper orientation.

When the surgeon has navigated the guide tube to the desired orientation and orthogonal distance from the point palpated on the medial cortex, he uses the guide tube to guide the drill for drilling the second k-wire.

When the surgeon can now indicate, such as by depressing the foot pedal, that he has completed the step of navigating the second k-wire. The system will record the angle and position of the second k wire and then proceed to the next screen.

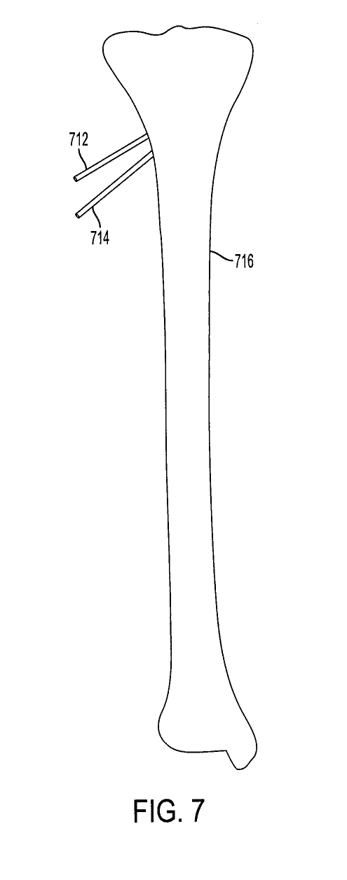

At this point, as shown in FIG. 7, there are two k-wires 712, 714 extending from the lateral side of the tibia 716, defining respectively, the varus-valgus angle of the first and second wedge cuts.

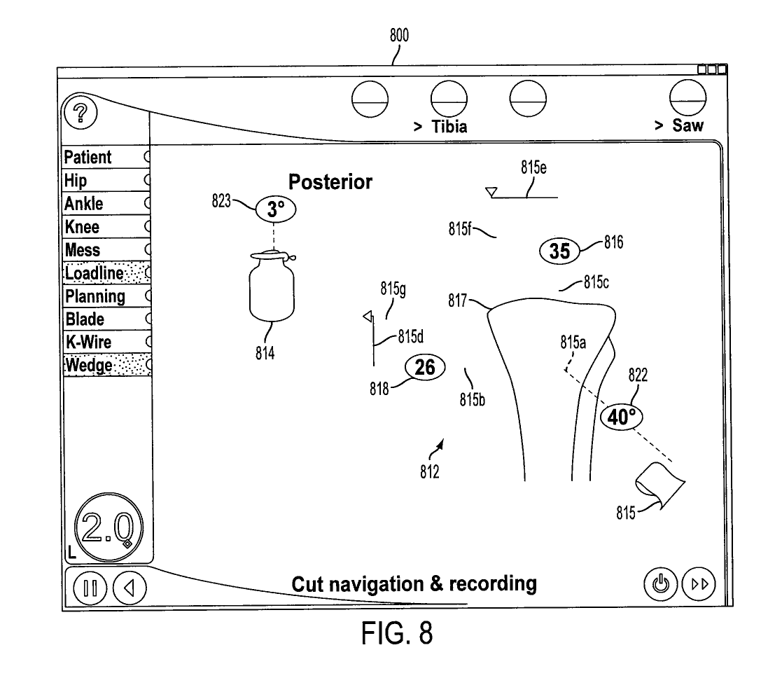

In the next screen, illustrated in FIG. 8, the surgical navigation system will be used to track the sagittal saw for making the first planar wedge cut. The surgeon mounts a marker on the sagittal saw. The surgical navigation system is preprogrammed with (or taught) information defining the plane of the saw blade as well as the position of the tip of the saw blade relative to the marker mounted on it. Screen 800 includes a frontal view of the tibia 812 as well as a pictorial rear view 814 of the saw. The frontal view also shows a side view representation of the saw 815, including the blade and particularly the tip 815a of the blade.

The frontal view shows several relevant pieces of information for navigating the first saw cut. Specifically, oval 816 shows the horizontal distance from the tip of the saw blade to the point palpated on the medial cortex and oval 818 shows the vertical distance from the tip of the saw blade to the point palpated on the medial cortex. This information is redundantly shown graphically by the representation of the saw 815 in the frontal view. Particularly, the view of the saw 815 shows the tip of the saw 815a relative to the point palpated on the medial cortex, which is represented by red point 817. It also includes dashed horizontal and vertical lines 815b and 815c drawn from the blade tip 815a to further lines 815d and 815e which, respectively, graphically show the distance from the blade tip 815a to the point palpated on the medial cortex 817. The lengths of lines 815b, 815c, 815d, and 815e change as the blade tip moves relative to the point palpated on the medial cortex. Thirdly, the angle of the saw relative to the tibial plateau is shown in oval 822 as well as redundantly by the graphical representation of the saw itself.

The surgeon typically will want to stop the cut about 5 to 10 mm short of the point palpated on the medial cortex.

The anterior-posterior slope of the saw blade is shown in the rear view 814 of the saw. The information is shown numerically in oval 823 as well as graphically by the tilt of the saw 814. Generally, the surgeon wants this angle to be about zero (measured relative to the tibial plateau). However, the particular anterior-posterior slope is less important than assuring that the anterior-posterior slope of the first and second cuts are the same. Otherwise, after the wedge is removed and the hinge bent to close the gap created by the removed bone wedge, the two surfaces will not mate well.

In any event, in accordance with the present invention, the surgeon rests the blade of the sagittal saw flat against and below the first k-wire thus assuring that the varus-valgus angle of the cut is the varus-valgus angle of the k-wire. In a preferred embodiment of the invention, the anterior-posterior slope of the cut is controlled free hand by the surgeon, without a cutting guide or other guide means (other than whatever added stability is provided by the ability to lay the blade on the k wire). The surgeon also controls the depth of the cut free hand using the screen of FIG. 8 to keep track of the orientation and depth of the cut.

It has been found that, even though the anterior-posterior slope and the depth of the cut are controlled manually free hand by the surgeon in this technique, it is very easy to make accurate cuts using this technique. Further, the technique is much faster since there is no need to mount a complex cutting jig requiring multiple screws or pins to affix in three degrees of freedom for each cut. Instead, only a single k wire is mounted for each cut, it is navigated in only two degrees of freedom, namely, varus-valgus angle and height (as previously noted, there also may be a rough check of rotational angle about the mechanical axis of the tibia), and it requires a single drilling operation.

In the preferred embodiment of the invention, the ovals 816 and/or 818 may turn red when the depth of the cut is within a predetermined distance of the point palpated on the medial cortex, such as 10 mm, in order to more clearly alert the surgeon that he or she is approaching the medial cortex. Alternately or in addition, either or both of the pictorial representations 814 and 815 of the saw may turn red.

When the surgeon has finished making the desired cut, he may press the input pedal to indicate the completion of the first wedge cut.

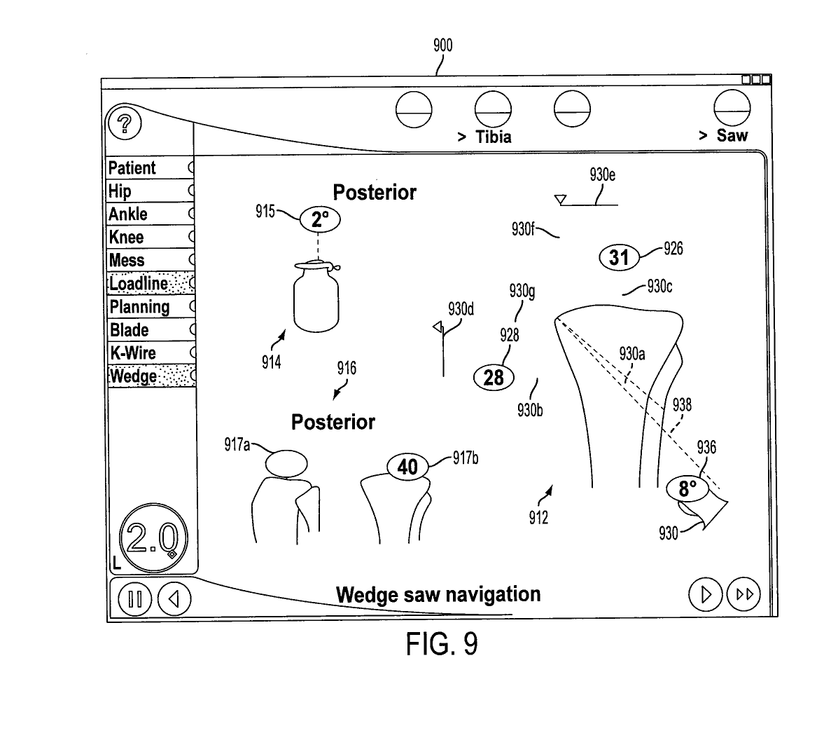

The surgical navigation system then switches to a new display screen such as shown in FIG. 9 that will be used for navigating the second wedge cut. This view may be considered to comprise three different portions, namely, a frontal view of the tibia 912, a rear view of the saw 914, and a small pair of views 916 comprising a sagittal view and a frontal view of the tibia. This last view 916 shows only the previously recorded anterior-posterior slope of the first cut in the oval 917a above sagittal (left hand) view and the previously recorded varus-valgus angle of the first kwire in the oval 917b above the frontal (right hand) view. Note that these two ovals 917a and 917b are shaded to indicate that they display recorded, unchanging data.

In FIG. 9, the information shown by the rear view of the saw 914 is essentially the same as that discussed above with respect to the corresponding portion of the screen of FIG. 8, namely the anterior-posterior slope of the saw blade. It is shown by the tilt of the saw 914 as well as numerically in oval 915.

Just as in the screen of FIG. 8, a side view graphical representation of the saw is shown at 930 in the frontal view. Also, in the frontal view 912, the horizontal and vertical distances of the tip of the saw blade to the point palpated on the medial cortex is shown in ovals 926 and 928, respectively. It is redundantly shown by lines 930b, 930c, 930d, 930e, 9390f, and 930g, just as in screen 800 of FIG. 8. The wedge angle is shown numerically in oval 936 and then redundantly graphically by the side view graphical representation 938 of the saw blade.

Furthermore, one or more of the saws 914, 930 and/or the ovals 926, 928, 936 may change colors when the tip of the saw blade is within a predetermined distance of the point palpated on the medial cortex.

As described above with respect to the first wedge cut, the surgeon lays the saw blade flat on the second k-wire in order to control the varus-valgus angle of the second cut. The surgeon controls the anterior-posterior slope of the cut as well as the depth of the cut freehand.

After the second cut is completed, the surgeon can then remove the bone wedge and proceed with the operation in the conventional manner. Particularly, and in short, the surgeon removes the wedge, bends the bone about the hinge to close the gap left by the wedge and then mounts, for example, a plate on the lateral side of the bone with screws to hold the bone in that position.

The methods and apparatus of the present invention are an improvement over prior art because it is much quicker and simpler for the surgeon. There is no need to navigate and mount a cutting jig in three degrees of freedom simultaneously. Rather, the k-wires are essentially navigated only in varus-valgus angles and height (and with an optional rough navigation of angular orientation about the mechanical axis of the tibia). Then when the surgeon is ready to make the cut with the sagittal saw, the varus-valgus angle and height are already set by the k-wire and the surgeon merely needs to manually control the anterior-posterior slope of the cut and the depth of the cut.

Another advantage of the method and apparatus of the present invention is that it is less tedious in that the surgeon navigates and mounts both k-wires using the guide tube 410 before making any cuts. Therefore, the marker can remain on the guide tube for navigating both k wires sequentially. In the prior art methods and apparatus, the cutting jig was navigated and mounted for the first cut. Then the cut was made with the sagittal saw, which typically requires the marker to be removed from the cutting jig and placed on the saw. Then the surgeon would need to go back and remount the marker on the cutting jig in order to navigate the cutting jig for the second cut. Finally, the surgeon would have to then remount the marker back on the sagittal saw in order to make the second cut. This was a laborious process of switching markers and instruments that the present invention has greatly simplified.

Another advantage of the present invention is that, if the k-wires are drilled into the bone to the point where they cross each other in the frontal view, they will provide extra safety in that they will prevent the saw from sawing past the point where the two k-wires cross in the frontal plane. That is, the surgeon cannot drill too far into the bone while resting the drill on one of the k-wires because the tip of the saw blade will hit the other of the two k-wires, thus preventing it from being further inserted past the desired safety point (i.e., 5 to 10 millimeters laterally of the medial cortex).

The invention has been described above in connection with the Wagner technique. However, this invention can be easily adapted for use in the Coventry technique. The mounting of two k-wires and navigating the varus-valgus angle of the saw by resting it on the k-wires would be largely the same. The screen displays preferably are slightly adapted to show different information. For instance, in the Coventry technique, in which the first cut is made parallel to the tibial plateau, it is important to assure that the second cut will be completely above the patellar insertion point. Whether the second cut will be completely above the patellar insertion point is a function of the wedge angle and the height of the first cut. Accordingly, the display might show in addition or instead of some of the information discussed above in connection with the Wagner technique, whether the first cut is at a height that will guarantee that the second cut will be completely above the patellar insertion point. This may be displayed numerically simply as the distance above or below the patellar insertion point of the second cut.

Also, the HTO procedure is merely one example of a surgical procedure in which the present invention can be implemented. The invention can be applied to many different surgical procedures involving the cutting of bones and the like.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A method of using a surgical navigation system for positioning a saw for making a cut in a bone at a particular orientation and position, said method comprising the steps of:
(1) navigating a guide for a longitudinal member relative to the bone to a first position in a first translational degree of freedom and first orientation in a first angular degree of freedom as a function of a desired position and orientation for a first cut in the bone;
(2) mounting a first longitudinal member to said bone using said guide positioned in said first position and first orientation as a guide; and
(3) using said saw to make said first cut in said bone while laying said saw against said mounted first longitudinal member, whereby said first longitudinal member controls the position and orientation of said first cut in said first angular degree of freedom and said first translational degree of freedom.

2. The method of claim 1 wherein, in step (3), the saw is controlled freehand in at least one other degree of freedom.

3. The method of claim 2 wherein, in step (3), the depth of the cut is controlled freehand.

4. The method of claim 1 further comprising the steps of:
(4) navigating said guide relative to the bone to a second position in said first translational degree of freedom and second orientation in said first angular degree of freedom as a function of a desired position and orientation of a second cut in the bone;
(5) mounting a second longitudinal member to said bone using said guide positioned in said second position and second orientation as a guide; and
(6) using said saw to make said second cut in said bone while resting said saw on said mounted second longitudinal member, whereby said second longitudinal member controls the position and orientation of said second cut in said first angular degree of freedom and said first translational degree of freedom.

5. The method of claim 4 wherein steps (1), (2), (4), and (5) are performed prior to steps (3) and (6).

6. The method of claim 1 wherein step (1) comprises displaying on a monitor in real time the position and orientation of said guide relative to said bone in at least said first angular degree of freedom and said first translational degree of freedom.

7. The method of claim 6 wherein step (3) comprises navigating said saw with said surgical navigation system; and displaying on said monitor said orientation of said saw in said first angular degree of freedom and said first translational degree of freedom.

8. The method of claim 7 wherein step (3) further comprises displaying on said monitor said orientation of said saw in at least a second angular degree of freedom.

9. The method of claim 8 wherein step (3) further comprises displaying on said monitor a distance of said cut from a particular point on said bone.

10. The method of claim 1 wherein said guide comprises a tube within which said longitudinal member can be inserted and step (2) comprises inserting said longitudinal member in said tube.

11. The method of claim 1 wherein said guide comprises a drill for drilling said longitudinal member into said bone and wherein step (2) comprises using said drill to drill said longitudinal member into said bone.

12. The method of claim 1 wherein step (1) further comprises navigating said guide in a third angular degree of freedom.

13. The method of claim 12 wherein step (1) comprises the step of:
(1.1) displaying on a monitor in real time the position and orientation of said guide relative to said bone in at least said first angular degree of freedom and said first translational degree of freedom; and
(1.2) displaying on said monitor whether or not said guide is oriented within a predetermined range in said third angular degree of freedom.

14. A method of using a surgical navigation system for navigating first and second cuts for cutting a wedge out of a tibia in a tibial osteotomy procedure, said method comprising the steps of:
(1) navigating a guide relative to said tibia to a desired varus-valgus angle and height for a first wedge cut;
(2) mounting a first longitudinal member to said tibia using said guide positioned at said desired varus-valgus angle and height as a guide;
(3) recording in said surgical navigation system the varus-valgus angle and height of said longitudinal member by recording the varus-valgus angle and height of said guide as it is positioned over said first longitudinal member after said first longitudinal member is mounted;
(4) navigating said guide relative to said tibia and said first longitudinal member to a desired varus-valgus angle and height of a second wedge cut;
(5) mounting a second longitudinal member to said tibia using said guide positioned at said desired varus-valgus angle and height for said second wedge cut as a guide;
(6) navigating said sagittal saw to make said first wedge cut in said tibia while laying said saw against said mounted first longitudinal member, whereby said first longitudinal member controls the varus-valgus angle and height of said first wedge cut; and
(7) navigating said sagittal saw to make said second wedge cut in said tibia while resting said saw on said mounted second longitudinal member whereby said second longitudinal member controls the varus-valgus angle and height of said second wedge cut.

15. The method of claim 13 wherein step (1) comprises displaying on a monitor of said surgical navigation system in real time the varus-valgus angle of said guide relative to said bone and an orthogonal distance of a longitudinal axis of said guide relative to a point palpated on the medial cortex of said tibia.

16. The method of claim 15 wherein step (4) comprises displaying on said monitor in real time the varus-valgus angle of said guide relative to said mounted first longitudinal member and an orthogonal distance of a longitudinal axis of said guide relative to a point palpated on the medial cortex of said tibia.

17. The method of claim 16 wherein step (6) comprises:
(6.1) navigating said saw relative to said tibia with said surgical navigation system; and
(6.2) displaying on said monitor the varus-valgus angle and anterior-posterior slope of said saw relative to said tibia, and a distance of said saw to the medial cortex of said tibia.

18. The method of claim 17 wherein step (7) comprises the steps of:
(7.1) navigating said saw relative to said tibia with said surgical navigation system; and
(7.2) displaying on said monitor the varus-valgus angle and anterior-posterior slope of said saw relative to said tibia, and a distance of said saw to a point palpated on the medial cortex of said tibia.

19. The method of claim 14 wherein said guide comprises a tube within which said longitudinal member can be inserted and step (2) comprises inserting said longitudinal member in said tube.

20. The method of claim 14 wherein said guide comprises a tube within which said longitudinal member can be inserted and step (2) comprises inserting said longitudinal member in said tube.

21. The method of claim 14 wherein step (1) further comprises navigating said guide to a desired angular orientation about a mechanical axis of said tibia.

22. The method of claim 15 wherein step (1) further comprises navigating said guide to a desired angular orientation about a mechanical axis of said tibia and displaying on said monitor whether or not said guide is oriented within a predetermined range of said desired angular orientation about said mechanical axis of said tibia.

23-29. (canceled)
